(19) United States
(12) Patent Application Publication
(10) Pub. No.: US 2008/0287909 A1
(43) Pub. Date: Nov. 20, 2008

(54) METHOD AND APPARATUS FOR INTRA-CHAMBER NEEDLE INJECTION TREATMENT

(76) Inventors: Raju R. Viswanathan, St. Louis, MO (US); Gareth T. Munger, St. Louis, MO (US); Ashwini K. Pandey, Marlborogh, MA (US)

Correspondence Address: Bryan K. Wheelock, Suite 400, 7700 Bonhomme, St. Louis, MO, 63105, US

(21) Appl. No.: 12/121,775

(22) Filed: May 15, 2008

Related U.S. Application Data
(60) Provisional application No. 60/938,700, filed on May 17, 2007.

(51) Int. Cl. A61M 37/00; A61B 17/00

(52) U.S. Cl. 604/506; 600/424

(57) ABSTRACT

A method is disclosed that enables intra-chamber needle injection treatment using a navigable interventional device. The method includes navigation steps to systematically inject cells, drugs, or other agents in diagnostically identified target tissues, such as ischemic heart wall tissues. Magnetic navigation of a specifically designed needle catheter permits safe access to remote structures of the heart and depth-adaptable needle injections.

### CROSS-REFERENCE TO RELATED APPLICATIONS

This application claims priority to U.S. Provisional Patent Application Ser. No. 60/938,700, filed May 17, 2007, the entire disclosure of which is incorporated herein.

### FIELD OF THE INVENTION

This invention relates to methods, devices, and system for the treatment of heart conditions and other conditions through the intra-cavity injection of cells, drugs, or other therapeutic agents delivered by a navigable interventional device comprising an extendable tip needle.

### BACKGROUND OF THE INVENTION

Interventional medicine is the collection of medical procedures in which access to the site of treatment is made by navigation through one of the subject's blood vessels, body cavities or lumens. Interventional medicine technologies have been applied to the manipulation of medical instruments, such as guide wires and catheters which contact tissues during surgical navigation procedures, making these procedures more precise, repeatable, and less dependent on the device manipulation skills of the physician. Remote navigation of medical devices is a recent technology that has the potential to provide major improvements to minimally invasive medical procedures. Several presently available interventional medical systems for directing the distal end of a medical device use computer-assisted navigation and a display means for providing an image of the medical device within the anatomy. Such systems can display a projection or cross-section image of the medical device being navigated to a target location obtained from an imaging system, such as x-ray fluoroscopy or computed tomography; the surgical navigation being effected through means, such as remote control of the orientation of the device distal end and proximal advance and retraction of the elongated medical device.

In a typical minimally invasive intervention, data are collected from a catheter or other interventional device instrumentation that are of significant use in treatment planning, guidance, monitoring, and control. For example, in diagnostic applications, right-heart catheterization enables pressure and oxygen saturation measurement in the right heart chambers, and helps in the diagnosis of valve abnormalities; left-heart catheterization enables evaluation of mitral and aortic valvular defects and myocardial disease. In electrophysiology applications, electrical signal measurements may be taken at a number of points within the cardiac cavities to map cardiac activity and determine the source of arrhythmias, fibrillations, and other disorders of the cardiac rhythm. Reliable systems have evolved for establishing arterial and venous access, controlling bleeding, and maneuvering catheters and catheter-based devices through the vascular tree to the treatment site.

The ventricle may respond erratically to atrial or nodal (atrioventricular) disturbances of rhythm. In the course of severe heart disease, such as coronary artery disease, ventricular tachycardia may occur. The beat is regular but may be so rapid that it interferes with normal cardiac filling and ejection and, therefore, results in either congestive failure, if prolonged, or in the development of a shock state, if severe and acute. Ventricular tachycardia is perhaps most important because it may be the forerunner of ventricular fibrillation, in which, as in atrial fibrillation, the contractions are widely erratic and ineffective, so that ventricular fibrillation interferes so much with ventricular function that circulation of the blood effectively ceases. Unless reversed within seconds or minutes, it is lethal.

Ischemic heart disease results from an insufficient supply of oxygen-rich blood to the myocardium; this condition typically results from a narrowing or blocking of a coronary artery by fatty and fibrous tissue, Death of a section of heart muscle results from a severe oxygen depletion; if the deprivation is insufficient to cause infarction, the effect may be angina pectoris. Progressive destruction of the myocardium occurs with repeated angina attacks. Both conditions can be fatal, as they can cause left ventricular failure or ventricular fibrillation inducing sudden cardiac death. Coronary bypass surgery or balloon angioplasty are indicated if medication and diet do not control progressive coronary heart disease and if the myocardial damage is not too extensive.

Radionuclide imaging provides a safe, quantitative evaluation of cardiac function and a direct measurement of myocardial blood flow and myocardial metabolism and thus, enables myocardial tissue characterization. Radionuclide imaging is used to evaluate the temporal progress of cardiac disease, hemodynamics, and the extent of myocardial damage during and after infarction and to detect pulmonary infarction following emboli. Technetium-99 is the radionuclide of choice in most phases of imaging. The recorded gamma ray data are acquired together with an ECG trace marking the cardiac cycle. These techniques are used to assess myocardial damage, left ventricular function, valve regurgitation, and, with the use of radionuclide potassium analogues, myocardial perfusion.

There are techniques that measure metabolism in the myocardium using the radiotracer method. Positron emission tomography uses positron radionuclides that can be incorporated into true metabolic substrates and consequently, can be used to follow the course of selected metabolic pathways, such as myocardial glucose uptake and fatty-acid metabolism. Myocardial perfusion imaging uses radioactive thallium to detect myocardial ischemia, myocardial infarction, and coronary artery disease. Injected intravenously, radioactive thallium is rapidly absorbed by the myocardium and is normally distributed evenly in heart muscle. Deficient blood flow to a portion of the myocardium is readily detectable by decreased thallium uptake in that area. Evidence of recent and not-so-recent myocardial infarcts will be visible, but most persons with coronary artery disease who have not had a previous infarction will have normal perfusion patterns when they are at rest. In such a patient, a thallium stress test is performed in which the substance is injected while the individual is exercising so that areas of transient ischemia can be identified and the patient treated to prevent myocardial infarction. Magnetic resonance imaging (MRI) also allows high resolution tomographic volumetric imaging of tissues.

At the end of 1998, almost simultaneously, one team of researchers (James A. Thomson) announced that it had isolated human embryonic stem (ES) cells and another (John Gearhart) announced that it had isolated human embryonic germ (EG) cells. These announcements gave rise both to the promise of great medical benefits and to contentious ethical and policy questions. The medical promise of these cells is the potential to provide an endless supply of transplantable tissue. The ethical and policy questions primarily concern the embryonic and fetal sources of these cells. The ES cells were isolated from a fertility clinic “spare embryos.” Such embryos, five to seven days old, are called blastocysts. The outer layer of the blastocyst is destined to become the placenta; the remainder of the blastocyst, called the inner cell mass, is destined to become the fetus. Embryonic stem cells are isolated from this inner cell mass. EG cells were isolated from five- to nine-week-old aborted fetuses. Such cells are referred to as embryonic germ cells, because they come from a small set of stem cells that were set aside in the embryo, prevented from differentiating, and were destined to evolve into eggs or sperm cells.

ES and EG cells share several remarkable properties. In principle the cells are of indefinite life. Whereas most cells divide a finite number of times and perish, ES and EG cells can be cultured to divide indefinitely. These cells are also plastic (pluripotent): they can turn into many cell types. All other cells present some degree of differentiation by turning into one or another type of cell, such as nerve or muscle or skin. It is likely that successfully directed ES and EG cell differentiation will be used in the future to generate specific, clinically transplantable tissues. ES cell research and clinical use relates to cloning. It is possible to use cloning, or somatic cell nuclear transfer (SCNT) to create a human being (reproductive cloning) or to create embryos as a source of ES cells. A patient can donate a tissue, and through SCNT, it is possible to create a source of ES cells with that patient's DNA. Consequently, this therapeutic cloning technique offers the potential to create tissues for transplantation that exactly match the recipient's tissues.

It is also noted that stem cells have also been found in unexpected places: in particular, “adult” stem cells are present in the striated cardiac muscle as muscular fiber precursors. These findings have opened new research vistas, as the use of such adult stem cells could circumvent the use of fetal cells or cloning.

Pilot animal studies have yielded very encouraging initial results indicating strong potential for the regeneration of ischemic heart tissue, Human trials are in process at at least one institution. These studies were conducted using mechanically navigated devices and mechanically actuated needles to inject therapeutic agents, including stem and modified stem cells, into the ischemic area of the heart.

### SUMMARY

The present invention relates to methods of navigating an interventional device in cavities of the body, such as a chamber of the heart, and delivering therapeutic agents at a series of target treatment points through needle injection.

### BRIEF DESCRIPTION OF THE DRAWINGS

FIG. 1-A shows a subject positioned in a projection imaging and interventional system for a minimally invasive procedure such as a left ventricle diagnostic and tissue regeneration therapeutic intervention;

FIG. 1-B illustrates an interventional device distal end being navigated through the subject's heart to collect diagnostic information, such as electrical activity in the left ventricle and perform injection therapy at selected tissue sites;

FIG. 2 presents a functional block diagram of a preferred embodiment of the present invention as applied to the interventional system of FIG. 1;

FIG. 3 shows a schematic endo-ventricular surface projection map view from the mitral valve down the long left ventricle axis;

FIG. 4 presents a schematic three-dimensional map of left ventricle target points;

FIG. 5 describes a schematic workflow pattern for the sequential injection treatment of a multiplicity of target sites within the left ventricle of the heart;

FIG. 6 illustrates improved approach angle to a target point in a magnetic navigation system; and

FIG. 7 presents a workflow of an embodiment of the method according to the present invention.

Corresponding reference numerals indicate corresponding parts throughout the several views of the drawings.

### DETAILED DESCRIPTION

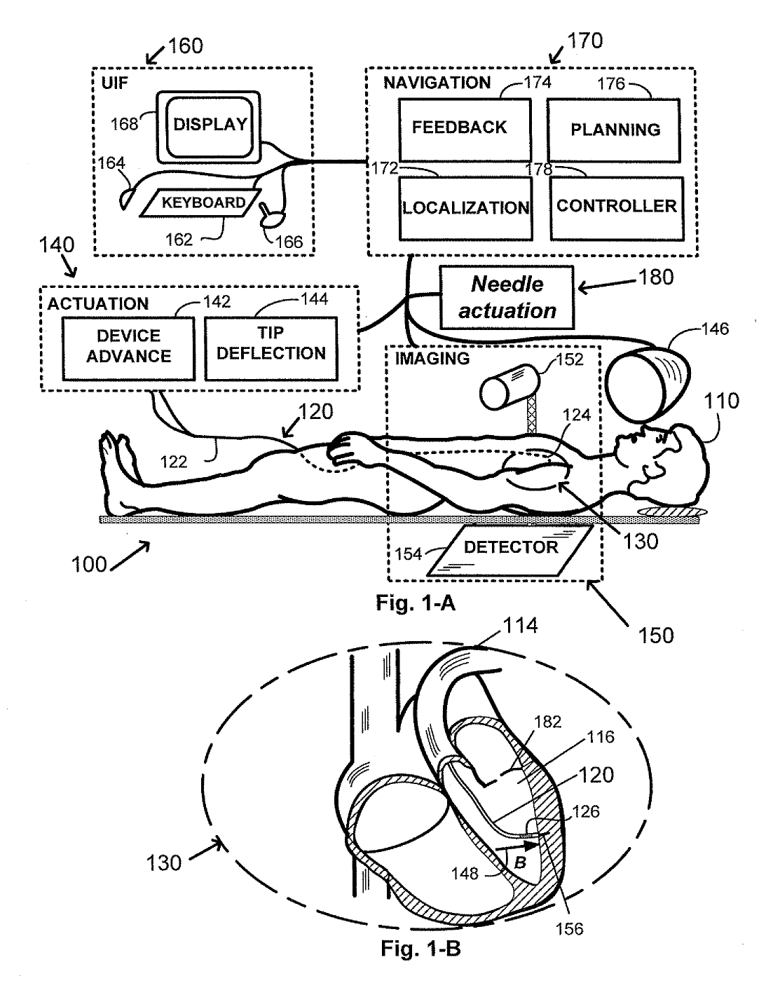

As illustrated in FIG. 1-A, a subject 110 is positioned within a remotely actuated, computer controlled interventional system, 100. An elongate navigable medical device 120 having a proximal end 122 and a distal end 124 is provided for use in the interventional system 100, and the medical device is inserted into a blood vessel of the subject, and navigated to an intervention volume 130. A means of applying force or torque to advance or orient the device distal end 124 is provided, as illustrated by actuation block 140 comprising a device advance/retraction component 142 and a tip deflection component 144. The actuation system for tip deflection may be one of (i) a mechanical pull-wire system; (ii) a hydraulic or pneumatic system; (iii) an electrostrictive system; (iv) a magnetic system; or (v) other navigation system, as known in the art.

For illustration of an embodiment, in magnetic navigation a magnetic field externally generated by magnet(s) assembly 146 orients a small magnet located at or near the device distal end 124. Real time information is provided to the physician by an imaging sub-system 150, for example, an x-ray projection imaging device comprising an x-ray tube 152 and an x-ray detector 154, to facilitate planning and guidance of the procedure. Additional real-time information may be supplied by use of a three-dimensional (3D) device localization sub-system, such as for example, comprising a set of electromagnetic wave receivers located at the device distal end (not shown), and associated external electromagnetic wave emitters (not shown); or other localization device with similar effect, such as an electric field-based localization system that measures local fields induced by an externally applied voltage gradient In the latter case the conducting body of a wire within the device itself carries the signal recorded by the tip electrode to a proximally located localization system.

The physician provides inputs to the navigation system through a user interface (UIF) sub-system 160 comprising user interfaces devices, such as a display 168, a keyboard 162, mouse 164, joystick 166, and similar input devices. Display 168 also presents real-time image information acquired by the imaging system 15o and localization information acquired by the three-dimensional localization system. UIF sub-system 160 relays inputs from the user to a navigation sub-system 170 comprising a 3D localization block 172, a feedback block 174, a planning block 176, and a controller 178. Navigation control sequences are determined by the planning block 176 based on inputs from the user, and also possibly pre-operative data, and localization data from a localization device and sub-system as described above, and processed by localization block 172, and real-time imaging, and additional feedback data processed by feedback block 174.

The navigation control sequence instructions are then sent to the controller 178 which actuates the interventional device 120 through actuation block 140 to effect device advance or retraction and device tip deflection. Other navigation sensors might include an ultrasound device or other device appropriate for the determination of distance from the device tip to the tissues, or for tissue characterization. Further device tip feedback data may include relative tip and tissues positions information provided by a local imaging system, predictive device modeling, or device localization system. Such device feedback in particular allows remote control of the intervention. In closed loop implementations, the navigation sub-system 170 automatically provides input commands to the device advance 142 and tip orientation 144 actuation components based on feedback data and previously provided input instructions; in semi-closed loop implementations, the physician fine-tunes the navigation control, based in part upon displayed information and possibly other feedback data, such as haptic force feedback feel. Control commands and feedback data may be communicated from the user interface 160 and navigation sub-system 170 to the device and from the device back to navigation sub-system 170 and the user through cables or other means, such as wireless communications and interfaces.

FIG. 1-B schematically shows the distal end 124 of the interventional device 120 having progressed through the aorta 114, through the aortic valve (not shown), and into the left ventricle 116. There the device distal end is magnetically navigated by an externally generated magnetic field B 148 that orients a small magnetically responsive element, such as magnet 126 positioned at or near the device distal end towards a series of points corresponding for instance, to pre-identified ischemic tissue areas. In diagnostic mode, the device collects functional information, such as electrical activity. As the device is localized in 3D through localization sub-system 172, the location and orientation of the distal end can be co-registered to pre-operative or intra-operative 3D anatomical image information. In such a manner, and for illustration of a typical application, after completion of cardiac chamber activity mapping, diagnostic information co-registered to 3D intra-operative image data is immediately available to navigation system 170 to automatically advance the interventional device to a series of points for agent injection therapy.

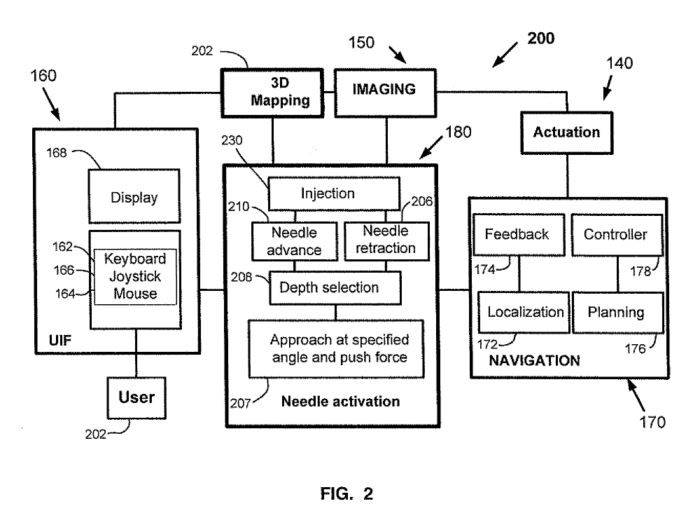

FIG. 2 shows a functional block diagram of a preferred implementation of the present invention generally indicated by numeral 200. The method for navigating a needle injection interventional device and effecting therapy is illustrated in FIG. 2, in reference to the components of the system block diagram shown in FIG. 1-A. It is noted that in specific implementations of the method, all or part of the functionalities indicated in FIG. 2 may be distributed among the other functional blocks of FIG. 1-A. Block 202, 3D mapping, represents the functional process of collecting diagnostic data and registering these data to a three-dimensional representation of the surface(s) of interest. In a typical application, pre-operative CT or MRI image data are available to navigation system 100, and mapping proceeds by advancing a diagnostic catheter to a series of points within the cavity of interest, collecting diagnostic data, and associating the data with the three-dimensional cavity representation. As such, the 3D mapping functionality shown in block 202 is usually assumed by the various navigation system blocks described in the context of FIG. 1-A. The devices designed to be operated by this method comprise a tip needle that can be proximally advanced and retracted over a range typically o to 10 mm. Proximal needle advance may be effected by the physician, or under computer control, either in semi-closed loop or closed-loop modes. The needle activation block 180 includes block 207 to specify the desired device approach angle to the local tissue surface at the target point.

In magnetic navigation implementations, the approach angle is realized by a corresponding time sequence of magnetic fields B(t) to be applied in the vicinity of the device distal tip to effect navigation along the pre-determined path.

In electrostrictively enabled navigation, electrostrictive elements at a number of locations along the device length shape the device, such that upon proximal advance contact to the tissue is effected at the prescribed approach angles. In mechanically enabled navigation systems, a time series of pull-wire and torque actions, as determined from a knowledge of the desired navigation path and mechanical device modeling, is applied to effect navigation. Specific implementations may use a combination of the techniques described, such as in the magnetic navigation of devices comprising electrostrictive or magnetostrictive elements.

The needle injection depth is determined by block 208 based on knowledge of the local anatomy, pre-acquired diagnostic information, possibly including nuclear imaging tissue characterization, and the selected approach angle. During the needle advance 210 into the tissue, the device navigation parameters are set to ensure continuous contact of the device distal end against the tissue wall, compensating for cardiac cycle motion forces, as known in the art. When the needle has been advanced by the pre-selected amount, therapeutic agent injection proceeds, 230, followed by needle retraction 206. In specific embodiments, block 180 comprises means for the automatic injection of one or more therapeutic agent(s): based on pre-identified target points and associated disease states, a list of target-point specific agents and associated amounts to be injected is automatically defined, and presented to the user for review and approval. Software editing means are provided, such as a graphical user interface, for the user to modify the list by editing either the agents or the amounts to be delivered. Proximally, a multiplexing injection port is provided that enables different agents to be delivered through the injection channel per the automatic protocol, as known in the art.

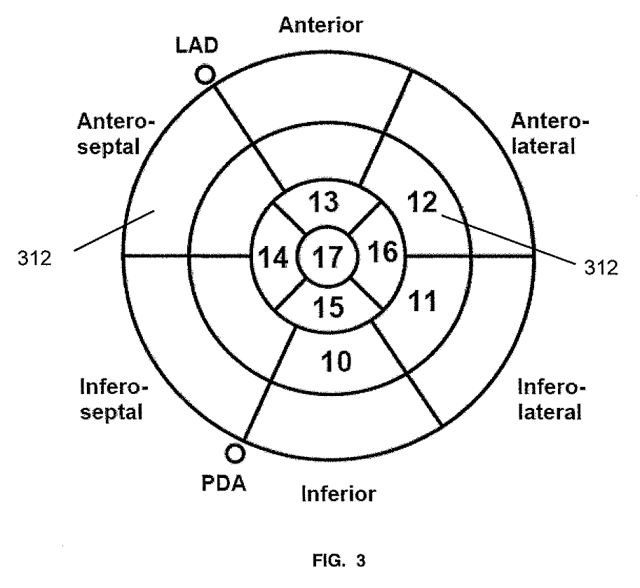

Methods have been developed to project a three-dimensional surface onto a two-dimensional surface; different projections types are obtained under different constraints. Projection methods that conserve angles are known as conformal. Other projection methods conserve surface areas. FIG. 3 presents such a planar schematic view of the left ventricle surface, as seen from the mitral valve 182 in the direction of the main ventricle axis. The ventricle surface has been divided in numbered sectors 312; areas that do not contain target therapy points are not labeled. This map and indicated target points are associated with the three-dimensional anatomy image representation in an unambiguous manner, such that navigation control sequences can be automatically generated by the system to lead the interventional device distal tip into contact with the pre-determined areas.

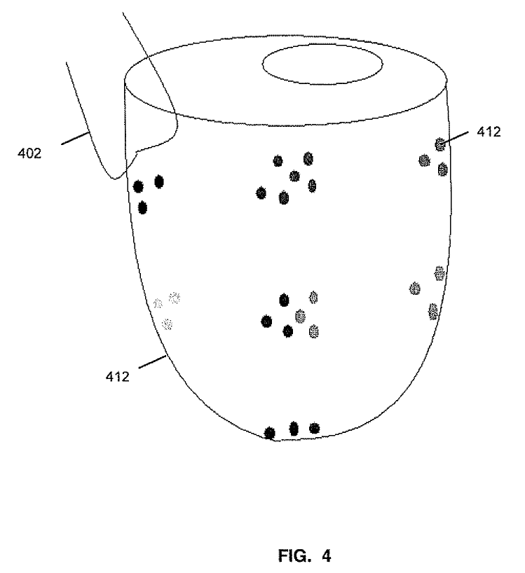

FIG. 4 shows a schematic 3D map of target points for injection therapy. Target points 412 are identified from the collection of diagnostic information, typically including the use of electrical activity mapping and/or nuclear imaging tissue characterization. The 3D diagnostic map is co-registered to 3D imaging data. In turn, the 3D diagnostic data can be projected, as shown in FIG. 3, and target points labeled on both 2D and 3D maps; as indicated above such marked points locations are known in 3D and the navigation system can automatically or semi-automatically advance a treatment device to each of the target points in turn.

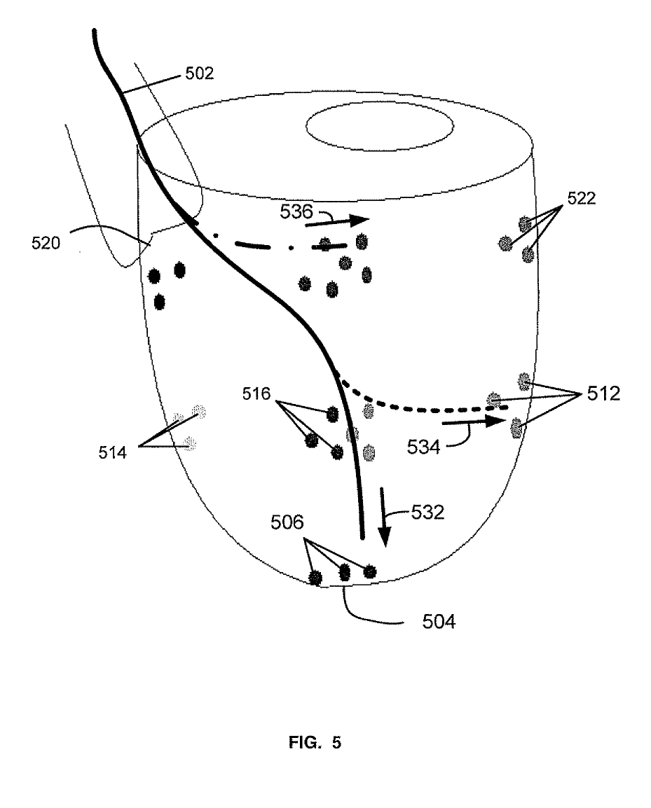

FIG. 5 schematically presents an interventional workflow for the sequential injection treatment of a multiplicity of tissue areas. Needle catheter 502 is advanced to the most remote location to be treated, as far as the ventricle apex 504 in the illustration of FIG. 5. Target points 506 that can be reached by locally reorienting the field and/or proximally rotating the catheter with minimal device advance/retraction are treated in sequence. Then the interventional device is retracted by the amount necessary for the next series of treatment points to be within close reach of the available device length extended in the chamber. According to this pattern, target point areas 512, 514, and 516 located at about the same distance from the aortic valve 520 are then treated in a next step. Then the interventional device is retracted to treat area 529, and the procedure continues until all areas targeted for treatment have been reached, therapeutic agent injected, and the device is retracted from the chamber. In the case of magnetic navigation, and as illustrated in FIG. 5, an externally applied magnetic field applied to a local volume around the device tip effect navigation to the selected target points, as shown by local fields B 532, 534, and 536 corresponding to different regions treated in sequence.

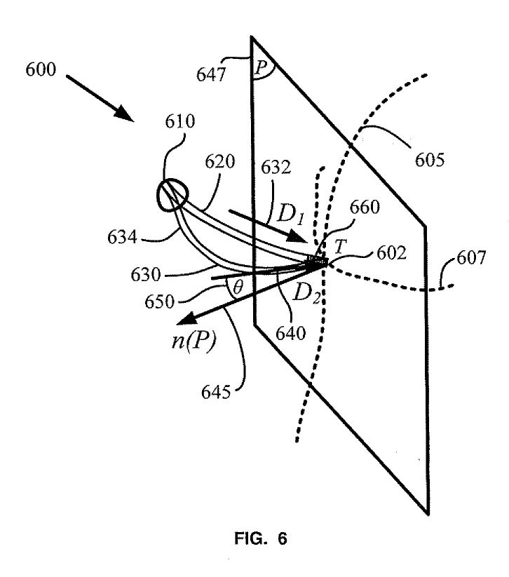

FIG. 6 illustrates two paths of approach to a target point T 602 to be treated by needle injection. Knowledge of the local topology of the heart, including orientation of local normal vector n(P) 645 and associated tangent plane P 647 at T are provided by the 3D mapping step, as for example, performed by the electrical activity mapping sub-system. This geometric information may also be derived from 3D imaging data, either pre- or intra-operative. The actual local heart surface at T is not necessarily planar; two lines 605 and 607 on the heart surface are shown. Given access to the heart chamber through opening or ostium 610 (for instance aortic valve) the shortest path 620 from the ostium to the target point leads to a large approach angle between n(P) and (−D₁) and glancing device incidence associated with direction vector D₁ 632. It is desirable in most situations for the interventional device and hence, the injection needle to be closer to or aligned with the normal n(P) to the local heart wall, as shown by approach path 634 following approach path D₂ 640 at T and associated approach angle θ 650: this geometry reduces the risks of mis-targeting associated with possible sliding of the catheter tip prior to needle advancement; as the heart moves during its cycle, and in particular during systole, significant forces are exerted on the tip that can lead to sliding and relative misplacement of the injection site. Additionally orthogonal or near orthogonal approaches, that is with approach angle θ 650 between n(P) and (−D₂) close to zero, enable more precise control of the needle injection depth into the wall tissues. Accordingly, and given knowledge of the interventional device mechanical properties, approach path 634 is outlined and associated control command sequences defined. As the interventional device is advanced under real-time imaging and localization control, fine adjustments are made to the control sequences to ensure contact occurs at the target point following the pre-defined path.

In specific situations, such as when the area to be treated is situated on the side of an elongated muscle fiber or “ridge,” it is desirable to define an alternative approach path that is at an angle with at least part of the local surface(s) around T. Once the alternative approach direction D₂ has been defined, the actuation proceeds as above to bring the device in tissue contact with the tip aligned with D₂. Knowledge of interventional device properties allows estimation of the best angle of approach θ given chamber geometry and the amount of force that can be applied. Specific designs will trade-off some amount of stiffness to enable more maneuverability; bending or buckling of the device at location 630 near device distal end 124 helps in achieving near orthogonal approaches in relatively small cavity volumes. Should the wall be relatively far away from ostium 610, it is possible to advance a sheath (not shown) in the cavity to provide support for the interventional device. In magnetic navigation system, direction D₂ would be that of a small tip magnet 660 upon wall contact, as achieved by a specific corresponding sequence of magnetic field orientations.

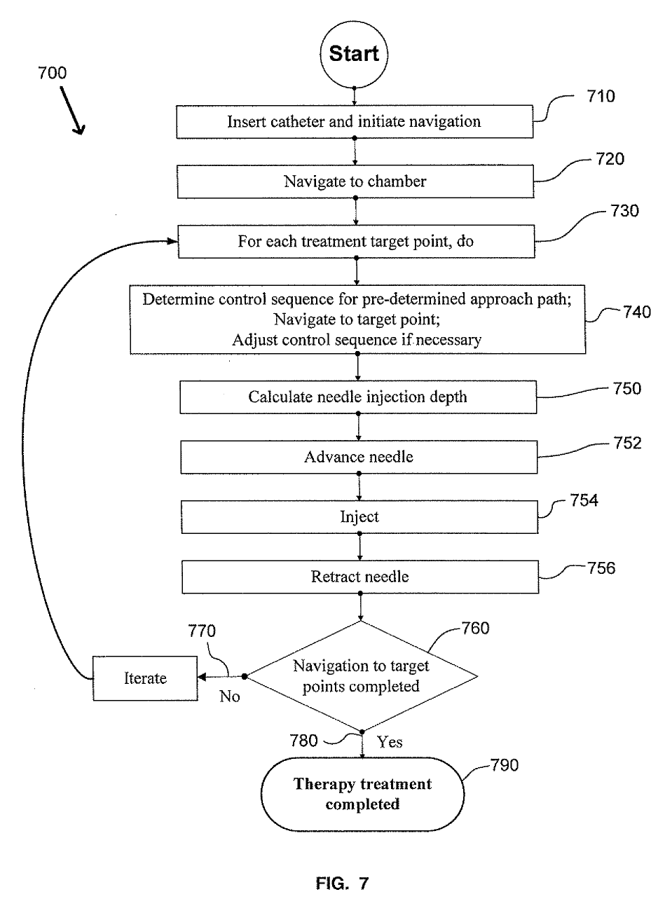

FIG. 7 presents a flow-chart for one embodiment of the method of the present invention, as generally indicated by numeral 700. Following vessel insertion, 710, the device is navigated to the chamber of interest 720. There the method iterates over loop 730 for each therapy target point. For each selected point the path of approach is determined, usually by the prescription of two angles characterizing approach path D₂ with respect to the local tangent plane P. These two angles are respectively approach angle θ and the angle between the projection of D₂ onto P and a reference axis within P. The associated control sequence is generated and fine-tuned as the device is navigated to contact, 740. The needle injection depth in the tissue is calculated as a function of the available diagnostic information, local tissue depth, and angle of incidence for approach path D₂, 750. Such diagnostic information could be available for example, from a CT scan or from a combination of CT and Positron Emission Tomography (PET) scans. Modern scanners combining both CT and PET modalities in three dimensions have now become available. PET scans are typically more coarse-grained (have fewer photon counts and lower spatial resolution) than CT scans.

The integrated three dimensional CT/PET volume data from such a dual-mode scanner can be imported into the remote navigation system (usually operated together with an X-ray imaging system for visualizing interventional devices in the anatomy) and registered to the latter through X-ray/3D registration. The diseased region can be identified coarsely in the PET data, and the definition of the diseased region can be refined through the use of 3D electro-anatomic mapping with a suitable localization and mapping system. The mapping system gathers data for and constructs an electrical map of a cardiac chamber based on the 3D coordinates and corresponding electrical signal propagation information at a set of distinct cardiac wall locations covering the desired region. Thus, one can construct a general map of the chamber and refine it locally within the region indicated on the (coarse) PET scan as a diseased region (perhaps with scar tissue). The refinement can be performed under manual control of the remote navigation system, or it can be automatically performed by the remote navigation system as it steers the catheter or medical device to a sequence of map-refinement target locations. Once the refined map is available, injection targets can be identified on the cardiac (endocardial) wall.

The injection needle is subsequently advanced, 752, the therapeutic agent injected, 754, and the needle retracted, 756, and the method proceeds to decision block 760. If the treatment of all target points are not completed, branch 770, the method iterates over blocks 740 to 756. Otherwise, branch 780, this therapy stage of the intervention is complete and the method terminates, 790.

The injection needle design takes into account navigation parameters, including target body cavity, required turn radius, force transmissible through the device to its distal end and associated pressure function of the needle tip area, and other relevant factors. Different navigation enabling technologies, such as mechanical pull-wire, electrostrictive, magnetic, and other, will lead to different constraint parameters and corresponding needle designs. Further, treatment of different cavities, as in for example access to different chamber of the heart, will impose specific set of requirements. For a given device turn radius and near-tip buckling properties, it is possible to calculate the distance from a given target point at which mechanical support is required for an optimal approach path. When the local chamber anatomy is such that the device extended length in the chamber exceeds that length, it is possible to insert a support sheath partially into the chamber to achieve improved navigation.

The advantages of the above described embodiments and improvements should be readily apparent to one skilled in the art, as to enabling intra-chamber needle injection treatment using a navigable interventional device. Additional design considerations may be incorporated without departing from the spirit and scope of the invention. Accordingly, it is not intended that the invention be limited by the particular embodiments or forms described above, but by the appended claims.

What is claimed is:

1. A method of performing an intra-cavity needle injection therapeutic procedure using a remotely actuated interventional navigation system, the method comprising:
(i) identifying a set of injection therapy target points within a subject's body cavity;
(ii) navigating an interventional device comprising a needle tip to the body cavity;
(iii) selecting an injection therapy target point from the identified set;
(iv) navigating the interventional device to the selected injection therapy target point;
(v) advancing the needle from the device tip into the tissue at the selected injection therapy target point;
(vi) delivering a therapeutic agent through the advanced needle; and
(vii) iterating over steps (iii) to (vi) to complete the therapeutic procedure.

2. The method of claim 1 further comprising the steps of determining for the device tip an approach path to and a contact angle at the selected injection therapy point and navigating the interventional device to the selected injection therapy point so that the approach path and contact angle are essentially as determined.

3. The method of claim 1 further comprising determining a tissue depth at the selected target point.

4. The method of claim 1 further comprising determining a needle injection depth for the selected target point.

5. The method of claim 3 further comprising determining a needle injection depth based on determined tissue depth at the selected injection therapy target point.

6. The method of claim 1 wherein the step of advancing the needle from the device tip into the tissue comprises proximally advancing an interventional device elongated component comprising a distal end and a proximal end;

7. The method of claim 1 wherein the step of advancing the needle from the device tip into the tissue comprises proximally activating a distally located needle advance actuator.

8. The method of claim 7 wherein the actuator is selected from the group consisting of (i) an electrical advancer; (ii) an electrostrictive advancer; and (iii) a mechanical advancer.

9. The method of claim 1 wherein the navigating comprises magnetically navigating the interventional device.

10. The method of claim 1 wherein the navigating comprises mechanically navigating the interventional device.

11. The method of claim 1 wherein the navigating comprises electrostrictively navigating the interventional device.

12. The method of claim 2 wherein the step of determining an approach path comprises using knowledge of the interventional device mechanical properties and knowledge of the cavity geometry.

13. The method of claim i wherein the step of identifying a set of injection therapy target points within a subject's body cavity comprises:
(i) identifying target points from a three-dimensional positron emission tomography image data set; and
(ii) registering the three-dimensional positron emission tomography image data set to the navigation system coordinates.

14. The method of claim 13 further comprising refining the set of target points identified from the three-dimensional positron emission tomography image data by:
(i) navigating a localized interventional device to the set of identified target points;
(ii) collecting diagnostic data at a second set of points comprising the identified target points and additional points in their vicinity;
(iii) characterizing the disease state for the second set of points based on collected diagnostic data, and defining a refined set of target points based on disease state characterization data.

15. The method of claim 1 wherein the step of navigating the interventional device to the selected injection therapy target point further comprises localizing the device tip in three-dimension using a localization system providing tip position and orientation information.

16. The method of claim 15 wherein the navigating the interventional device to the selected injection therapy target point is performed automatically using localization data co-registered with the navigation system coordinate system.

17. The method of claim 1 wherein the step of navigating the interventional device to the selected injection therapy target point further comprises using a computational model of the interventional device to effect navigation.

18. The method of claim 17 further comprising the step of collecting additional diagnostic data at and near the points reached by the interventional device to confirm or modify the set of injection therapy target points.

19. A method of injecting a therapeutic agent at a target point within a subject body cavity, the method comprising:
(i) navigating an interventional device comprising a distal tip injection needle to the vicinity of the target point;
(ii) navigating the interventional device distal tip to contact the target point;
(iii) advancing the distal tip injection needle in the tissue; and
(iv) injecting the therapeutic agent.

20. The method of claim 19 further comprising the steps of determining for the device tip an approach path and a contact angle at the target point and navigating the interventional device to the target point so that the approach path and contact angle are essentially as determined.
